(19) United States
(12) Patent Application Publication
(10) Pub. No.: US 2010/0080821 A1
(43) Pub. Date: Apr. 1, 2010

(54) Energy Compositions

(75) Inventor: Peter Spiegel, Los Angeles, CA (US)

Correspondence Address: Marc Van Dyke, 123 N.W. 13th Street, Suite 221, Boca Raton, FL, 33432-1619, US

(73) Assignee: SYLMARK, INC., Lake Balboa, CA (US)

(21) Appl. No.: 12/569,920

(22) Filed: Sep. 30, 2009

Related U.S. Application Data
(60) Provisional application No. 61/101,535, filed on Sep. 30, 2008.

(51) Int. Cl. A61K 36/06; A61P 43/00

(52) U.S. Cl. 424/195.15

(57) ABSTRACT

Described herein are caffeine-free compositions that provide a sense of energy and well-being. In one aspect, the compositions include a blend of natural herbs or herbal extracts such as at least one Schizandra, at least one Rhodiola, an eleutheroside, at least one Cordyceps, at least one Lycium (genus of goji), and at least one Ribes to revitalize one's state of well being. Additional herbs, herbal extracts, sweeteners, vitamins, minerals, and additional nutrients may be added to the composition for additional benefits and improved taste. In some embodiments, the composition is provided as a beverage, e.g. a carbonated beverage.

### CROSS-REFERENCE TO RELATED APPLICATION

This application claims priority to U.S. provisional application No. 61/101,535, filed on Sep. 30, 2009. This application is hereby incorporated by reference in its entirety for all of its teachings.

### RELATED ART

Over the past decade, energy formulations such as energy powders and energy drinks have become wildly popular. For example, in 2006 alone, the sports energy drink industry generated $3.7 billion dollars. These formulations have been modified to use in all facets of life such as pre or post workout supplements, in recreational settings such as bars or clubs, or to generally provide more energy during the work day. However, due to the presence of large amounts of caffeine and sugars in these compositions, the compositions are not always attractive.

In 2006, the Journal of Analytical Toxicology published a study where a team of researchers analyzed the caffeine content of 10 popular energy drinks and found concentrations as high as 141 milligrams per 16-ounce can. While the Food and Drug Administration does not regulate the amount of caffeine in soft drinks or energy powders, agency guidelines for colas suggest no more than 68 milligrams per 12-ounce serving.

While some dismiss these statistics, high caffeine intake over long periods of time may lead to serious health problems. Some concerns regarding high caffeine and sugar levels include fostering an unhealthy dependence on caffeine, increasing one's tendency to gain weight, increasing one's blood pressure or leading to high blood pressure, increasing one's chance for cardiovascular disease, heart arrhythmia, and increasing one's risk for anxiety disorders.

### SUMMARY OF EMBODIMENTS

Described herein are caffeine-free compositions that provide a sense of energy and well-being. In one aspect, the compositions include a blend of natural herbs or herbal extracts such as at least one species of Schizandra, at least one species of Rhodiola, an eleutheroside, at least one species of Cordyceps, at least one species of Lycium (genus of goji), and at least one species of Ribes to revitalize one's state of well being. Additional herbs, herbal extracts, sweeteners, vitamins, minerals, and additional nutrients may be added to the composition for additional benefits and improved taste.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION OF EMBODIMENTS

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a Rhodiola” includes mixtures of two or more such species, and the like.

“Optional” or “optionally” means that the subsequently described event or circumstance can or cannot occur, and that the description includes instances where the event or circumstance occurs and instances where it does not. For example, the phrase “optionally grape seed extract” means that grape seed extract may be optionally added to the composition.

The term “adaptogen” refers to a natural herb product that increases the body's resistance to stress, trauma, anxiety, and fatigue. All adaptogens contain antioxidants, but antioxidants are not necessarily adaptogens.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

Described herein are caffeine-free compositions that provide a sense of energy and well-being. The compositions utilize a blend of natural herbs and herbal extracts to revitalize one's state of well being. Additional herbs, antioxidants, herbal extracts, sweeteners, vitamins, minerals, and additional nutrients may be added to these compositions for additional benefits. The compositions described herein can be prepared into a variety of different formulations. In one aspect, the compositions can be formulated as a powder, a solid cake, a tablet, a gel, or a pre-made aqueous solution. In the case of the pre-made aqueous solution, it can include at least distilled water or filtered water and the compositions described below. In certain aspects, the water contains minimal to no electrolytes and little or no preservatives

The compositions can be used in a variety of different food products. In one aspect, the powder form of the compositions can be added to water or any aqueous beverage including tea, milk, various soft drinks, coffee, smoothies and other supplemental beverages. In one aspect, about 1 to 10, or 5 grams of the powder can be added to 4-8 oz of a beverage such as, for example, water, tea, milk, various soft drinks, coffee, smoothies, and other supplemental beverages. The compositions may be consumed as many times as needed, with one to four times a day being optimal.

In another aspect, the compositions can be added to and consumed with food. Once mixed with food, the composition provides additional antioxidants and nutrients to further supplement the food it is mixed with. For optimal results, the powder form of this composition may be consumed one to four times a day. In these aspects, the composition can be added to fruit and other food groups.

As described above, the compositions described herein have an abundance of antioxidants and nutrients. In one aspect, the compositions have a high Oxygen Radical Absorption Capacity (ORAC) value which may further enhance one's sense of well being. In one aspect, the composition's ORAC value ranges from 900 to 1500 per 5 grams of the composition. In yet another aspect, the compositions ORAC value ranges from 1300 to 1400 per 5 grams of composition. To further compare the compositions described herein to fruits and vegetables, the following fruits have ORAC values as shown in parentheses per 100 grams: blueberries (2,400), blackberries (2,036), cranberries (1,750), strawberries (1,540), raspberries (1,220), plums (949), oranges (750), red grapes (739), cherries (670), kiwi fruit (602), and white grapes (446).

In one aspect, the compositions described herein include at least one species of Schizandra, at least one species of Rhodiola, at least one eleutheroside, at least one species of Cordyceps, at least one species of Lycium, and at least one species of Ribes. The compositions provide an alternative to the conventional, caffeine containing energy drinks mentioned above by delivering these natural herbs and herbal extracts, which include Asian roots, to the body. Without wishing to be bound by theory, the compositions are advantageous because they provide one with energy without the negative side effects associated with conventional energy drinks.

In one aspect, the compositions include at least one species of Schizandra. For example, these species may include Schizandra chinensis, Schizandra glaucescens, Schizandra rubriflora, Schizandra rubrifolia, or any combination thereof. Schizandra has been used in Asia for over 5000 years because of its medicinal properties which include supporting mental alertness, liver health, kidney health, and skin health. Additionally, this genus of herb may have anti-bacterial, antioxidant, and anti-depressant properties. This genus is particularly known for possessing multiple flavors such as sourness, bitterness, sweetness, spiciness, and saltiness and is often termed the five-flavor fruit.

The major active ingredient of Schizandra are lignans called schizandrines. Schizandra increase activities of some enzymes that intervene in oxidative phosphorylation. For example, Schizandra chinensis effectively increases mental performance and physical working capacity in humans. Schizandrol A is one of the effective components in the dried fruit of Schizandra chinensis.

The amounts of Schizandra in the compositions may vary. However, in one aspect, the composition contains at least 1.5 wt % to 2 wt % Schizandra.

In another aspect, the compositions include at least one species of Rhodiola. For example, these species may include Rhodiola rosea, Rhodiola heterodonta, Rhodiola semenovii, or any combination thereof. Rhodiola grows primarily in high mountain regions of the Arctic and North Asia, and studies have shown that Rhodiola helps improve endurance, boost mood levels, and fight fatigue.

Species of Rhodiola such as, for example, Rhodiola rosea (Golden Root, Roseroot, Artic root), consists mainly of phenylpropanoids (rosavin, rosin, rosarin—all specific to R. rosea), phenylethanol derivatives (salidroside, rhodioloside, tyrosol), flavanoids (catechins, proanthocyanidines, rodiolin, rodionin, rodiosin, acetylrodalgin, tricin), monoterpenes (rosiridol, rosaridin), triterpenes (daucosterol, beta-sitosterol), and phenolic acids (chlorogenic, caffeic, hydroxycinnamic and gallic acid).

Rhodiola, used within the compositions, may be an extract. In this aspect, the Rhodiola extract is prepared by extraction, preferably of the roots, and/or of the low stem of the plant, by a solvent, followed optionally by a fractioning step enabling the active fraction(s) to be isolated. The solvent can be polar or non-polar. Examples of solvents include, but are not limited to, pentane, decane, cyclohexane, hexane, petroleum ether, monochloromethane, dichloromethane, chloroform, isopropanol, propanol, ethyl acetate, ethanol, methanol, acetone, butylene glycol, propylene glycol, pentylene glycol, glycerol, water, and any mixture of at least two of these solvents, particularly water-alcoholic or water-glycolic mixtures.

The amounts of Rhodiola in the compositions may vary. However, in one aspect the composition contains at least 1.5 wt % to 2 wt % Rhodiola. In one aspect, the Rhodiola within the composition contains at least 3% rosavins and 1% salidroside.

Eleutherococcus is a genus of about 38 species of shrubs which grow throughout Asia. Many members of this species contain ginseng or various chemicals which act in a similar manner to ginseng. The compositions may include at least one species of Eleutherococcus, such as Eleutherococcus senticosus which includes Eleutherococcus root, extracts thereof, active components thereof, or mixtures thereof. Eleutherococcus (Acanthopanax) senticosus has been referred to as Siberian ginseng. As used herein, the preferred common name now is eleuthero. Eleutherococcus is valued as an adaptogen and is believed to help combat fatigue. In one aspect, Eleutherococcus senticosus has been found to improve memory, boost general well-being, and aid in one's ability to manage stress.

Eleutherococcus or Acanthopanax senticosus (Russian Ginseng, Siberian Ginseng, Eleuthero, Devil's Shrub, Buisson du Diable, Touch-me-not, Wild Pepper, Shigoka, Acantopanacis senticosus) contains terpenoids (oleanolic acid), Eleutheroside A (daucosterol); Eleutheroside B (siringin); Eleutheroside B1 (isofraxidin); Eleutheroside B4 (sesamin); Eleutheroside D and E (heteroside siringoresinol); Eleutheroside C, G, I, K, L and M; phytosterols (β-sitosterol), polysaccharides (eleutherans), volatile oils, caffeic acid, coniferyl aldehyde, and sugars.

In one aspect, examples of species of Eleutherococcus useful herein include, but are not limited to, the following: Eleutherococcus baoxinensis, Eleutherococcus brachypus, Eleutherococcus cissifolius, Eleutherococcus cuspidatus, Eleutherococcus divaricatus, Eleutherococcus eleutheristylus, Eleutherococcus giraldii, Eleutherococcus henryi, Eleutherococcus higoensis, Eleutherococcus huangshanensis, Eleutherococcus hypoleucus, Eleutherococcus japonicus, Eleutherococcus lasiogyne, Eleutherococcus leucorrhizus, Eleutherococcus nanpingensis, Eleutherococcus nikaianus, Eleutherococcus nodiflorus, Eleutherococcus pilosulus, Eleutherococcus pseudosetulosus, Eleutherococcus pubescens, Eleutherococcus rehderianus, Eleutherococcus rufinervis, Eleutherococcus scandens, Eleutherococcus senticosus, Eleutherococcus seoulensis, Eleutherococcus sessiliflorus, Eleutherococcus setchuensis, Eleutherococcus setulosus, Eleutherococcus sieboldianus, Eleutherococcus simonii, Eleutherococcus spinosus, Eleutherococcus stenophyllus, Eleutherococcus trichodon, Eleutherococcus trifoliatus, Eleutherococcus verticillatus, Eleutherococcus wardii, Eleutherococcus wilsonii, Eleutherococcus xizangensis, or any combination thereof.

The amounts of Eleutherococcus in the compositions may vary. However, in one aspect the composition contains at least 1.5 wt % to 2 wt % Eleutherococcus.

Cordyceps helps maintain a healthy immune system and has been used in traditional Chinese medicine for its ability to restore energy, promote longevity, and improve one's quality of life. Cordyceps is a genus of fungi that includes about 400 described species. Some Cordyceps species are sources of biochemicals with interesting biological and pharmacological properties, like cordycepin; the anamorph of Cordyceps subsessilis (Tolypocladium inflatum) was the source of ciclosporin—a drug helpful in human organ transplants, as it suppresses the immune system.

In one aspect, the compositions include at least one species of Cordyceps. This at least one species may include, for example, Cordyceps bassiana, Cordyceps gunnii, Cordyceps ophioglossoides, Cordyceps sinesis, Cordyceps subsessilis, Cordyceps unilateralis, or any combination thereof. The amounts of Cordyceps in the composition may vary. However, in one aspect the composition contains at least 0.3 wt % to 0.5 wt % Cordyceps.

Lycium is a genus of the nightshade family (Solanaceae), containing about 90 species of plants native throughout much of the temperate and subtropical zones of the world. European herbalists have recognized Lycium for its medicinal properties since ancient times. In ancient texts, boxthorn (a species of Lycium) was described as a medicinal plant recommended as a treatment for sore eyes and inflammation. Various species of Lycium have been used in herbal teas, soups, juices, and alcoholic beverages.

In one aspect, the compositions include at least one species of Lycium such as, for example, Lycium barbarum, Lycium chinense, or any combination thereof. In this aspect, Lycium barbarum (Goji or Wolfberry) may be used within the composition. Without wishing to be bound by theory, Lycium barbarum (Goji or Wolfberry) may be used within the compositions to enhance energy levels, support kidney and liver function, build strong muscles and bones, reduce cardiovascular and inflammatory disease, reduce vision-related disease, and aid in neuroprotection.

The amounts of Lycium in the composition may vary. However, in one aspect the composition contains at least 3.0 wt % to 6.0 wt % Lycium.

Ribes is a genus of about 150 species of flowering plants. Ribes includes the currants, including edible currants such as blackcurrant, redcurrant, and whitecurrant, gooseberries, and many ornamental plants. Ribes including currants have been touted for their medicinal purposes.

In one aspect, the compositions include at least one species of Ribes. Examples include, but are not limited to, Ribes nigrum (blackcurrant), Ribes aciculare, Ribes alpestris, Ribes alpinum, Ribes altamirani, Ribes ambiguum, Ribes americanum, Ribes aureum, Ribes bracteosum, Ribes burejense, Ribes californicum, Ribes cereum, Ribes ciliatum, Ribes coloradense, Ribes curvatum, Ribes cynosbati, Ribes diacanthum, Ribes distans, Ribes divaricatum, Ribes echinellum, Ribes emodense, Ribes fasciculatum, Ribes fragrans, Ribes gayanum, Ribes giraldii, Ribes glaciale, Ribes glandulosum, Ribes glutinosum, Ribes grossularioides, Ribes henryi, Ribes hirtellum, Ribes hudsonianum, Ribes indecorum, Ribes inebrians, Ribes inerme, Ribes irriguum, Ribes japonicum, Ribes lacustre, Ribes lasianthum, Ribes laurifolium, Ribes laxiflorum, Ribes leptanthum, Ribes lobbii, Ribes longeracemosum, Ribes luridum, Ribes malvaceum, Ribes mandschuricum, Ribes maximowiczii, Ribes menziesii, Ribes meyeri, Ribes missouriense, Ribes montigenum, Ribes moupinense, Ribes multiflorum, Ribes nevadense, Ribes nigrum, Ribes niveum, Ribes odoratum, Ribes orientale, Ribes oxyacanthoides, Ribes petiolare, Ribes petraeum, Ribes pinetorum, Ribes procumbens, Ribes pulchellum, Ribes quercetorum, Ribes roezlii, Ribes rotundifolium, Ribes rubrum, Ribes sanguineum, Ribes setosum, Ribes silvestre, Ribes speciosum, Ribes spicatum, Ribes stenocarpum, Ribes tenue, Ribes triste, Ribes ussuriense, Ribes uva-crispa, Ribes viburnifolium, Ribes vilmorinii, Ribes viscosissimum, Ribes warszewiczii, Ribes wolfi, or any combination thereof.

In one aspect, the composition includes Ribes nigrum (blackcurrant). Blackcurrant contains high levels of vitamin C, potassium, phosphorus, iron, and vitamin B5. In addition, blackcurrant contains polyphenols/anthocyanins which potentially inhibit inflammatory mechanisms suspected to be at the origin of heart disease, cancer, microbial infections or neurological disorders like Alzheimer's disease. Major anthocyanins in blackcurrant include delphinidin-3-O-glucoside, delphinidin-3-O-rutinoside, cyanidin-3-O-glucoside, and cyanidin-3-O-rutinoside. Blackcurrant seed oil is also rich in many nutrients, such as gamma-linolenic acid (GLA), an essential fatty acid. It is theorized that essential fatty acids play a role in cardiac health and may improve brain function.

The amounts of Ribes in the compositions may vary. However, in one aspect the composition contains at least 25 wt % to 40 wt % Ribes.

In one aspect, the compositions described herein also possess one or more complex carbohydrates. The complex carbohydrate utilized herein can be an oligosaccharide, polysaccharide, and/or carbohydrate derivative, preferably an oligosaccharide and/or polysaccharide. As used herein, the term “oligosaccharide” means a digestible linear molecule having from 3 to 9 monosaccharide units, wherein the units are covalently connected via glycosidic bonds. As used herein, the term “polysaccharide” means a digestible (i.e., capable of metabolism by the human body) macromolecule having greater than 9 monosaccharide units, wherein the units are covalently connected via glycosidic bonds. The polysaccharides may be linear chains or branched. In one aspect, the polysaccharide has from 9 to about 20 monosaccharide units. Carbohydrate derivatives, such as a polyhydric alcohol (e.g., glycerol), may also be utilized as a complex carbohydrate herein. As used herein, the term “digestible” means capable of metabolism by enzymes produced by the human body. Examples of polysaccharides not within the definitions herein include resistant starches (e.g., raw corn starches) and retrograded amyloses (e.g., high amylose corn starches) since such polysaccharides are known to be non-digestible by the human body.

Non-limiting examples of complex carbohydrates used within the compositions include raffinoses, stachyoses, maltotrioses, maltotetraoses, glycogens, amyloses, amylopectins, polydextroses, and maltodextrins.

Maltodextrins are a form of complex carbohydrate molecule which is several glucose units in length. Without intending to be limited by theory, since maltodextrins are hydrolyzed into glucose in the digestive tract, they may be utilized as an extended source of glucose. Maltodextrins may be spray-dried carbohydrate ingredients made by controlled hydrolysis of corn starch. As is commonly known in the art, the dextrose equivalence (“DE”) of maltodextrins provides a good index of the degree of starch polymer hydrolysis. Preferred maltodextrins are those with a DE about 22 or less. Preferred maltodextrins for use herein are those with a DE of from about 15 to about 20, more preferably from about 16 to about 20.

When present, the amount of maltodextrin in the composition may vary. However, in one aspect the composition contains at least 20 wt % to 30 wt % maltodextrin.

In another aspect, additional sweeteners may be added to the compositions. These additional sweeteners may include both natural and artificial sweeteners. For example, high intensity sweeteners can also be sugar substitutes that reduce the net effective carbohydrate content of food while still providing a sweet taste. These high intensity sweeteners can be used in addition to, or in place of, sugar alcohols and fiber sweeteners. These sweeteners can be hundreds of times sweeter than sucrose, yet they contain many fewer calories than sucrose. High intensity sweeteners also have little to no effect on blood sugar levels. High intensity sweeteners include, but are not limited to, aspartame, alitame, neotame, cyclamates, saccharin, acesulfame, sucralose, neohesperidin dihydrochalcone, stevia sweeteners, glycyrrhizin, thaumatin, and the like, and mixtures thereof. Usually the amount of high intensity sweetener used is scant since it has such an intense sweetness.

In one aspect, stevia, in an herbal form or extract containing steviosides, may be added to the compositions. Stevia may be a desirable additive because of its sweet taste but negligible effect on blood glucose. It is an attractive natural sweetener for diabetics and others on a carbohydrate-controlled diet. When present within the composition, the amount of stevia varies depending on the sweetness desired. In one aspect, the composition contains at least 0.7 wt % to 1.0 wt % stevia.

The compositions may also contain additional components which are high in antioxidants. For example, Finnish pine (Pinus sylvestris) extract, grape seed extract, or both may be included within the composition. These extracts both contain similar active agents such as oligomeric proantocyanidins (OPC), cathecins and polyphenols. For example, the antioxidant properties of Finnish pine extract inhibits hydroxyl radicals about 10 times more efficiently than a portion of green tea and about 5 times more efficient that a daily dose (60 mg) of vitamin C.

In one aspect, the grape seed extract is 95% pure. Preferably, the composition contains at least 0.7 wt % to 1.0 wt % grape seed extract.

In another aspect, one or more flavoring agents are recommended for the compositions to enhance its palatability. Any natural or synthetic flavor agent can be used in the compositions described herein. For example, one or more botanical and/or fruit flavors may be utilized herein. As used herein, such flavors may be synthetic or natural flavors.

Particularly preferred fruit flavors are exotic and lactonic flavors such as, for example, passion fruit flavors, mango flavors, pineapple flavors, cupuacu flavors, guava flavors, cocoa flavors, papaya flavors, peach flavors, and apricot flavors. Besides these flavors, a variety of other fruit flavors can be utilized such as, for example, apple flavors, citrus flavors, grape flavors, raspberry flavors, cranberry flavors, cherry flavors, and the like. These fruit flavors can be derived from natural sources such as fruit juices and flavor oils, or may alternatively be synthetically prepared.

Preferred botanical flavors include, for example, tea (e.g. black and green tea), aloe vera, guarana, ginseng, ginkgo, hawthorn, hibiscus, rose hips, chamomile, peppermint, fennel, ginger, licorice, lotus seed, saw palmetto, sarsaparilla, safflower, St. John's Wort, curcuma, cardimom, nutmeg, cassia bark, buchu, cinnamon, jasmine, haw, chrysanthemum, water chestnut, sugar cane, lychee, bamboo shoots, vanilla, coffee, and the like.

The flavor agent can also comprise a blend of various flavors. If desired, the flavor in the flavoring agent may be formed into emulsion droplets which are then dispersed in the composition. Because these droplets usually have a specific gravity less than that of water and would therefore form a separate phase, weighting agents (which can also act as clouding agents) can be used to keep the emulsion droplets dispersed in the composition. Examples of such weighting agents are brominated vegetable oils (BVO) and resin esters, in particular the ester gums. Typically the flavoring agents are conventionally available as concentrates or extracts or in the form of synthetically produced flavoring esters, alcohols, aldehydes, terpenes, sesquiterpenes, and the like.

In a further aspect, the composition includes the formulation of Table 1. The amounts of each component are expressed as the weight of the processed ingredients.

The ratios provided for ingredients 1, 2, and 3 indicate the ratio of raw ingredients to powdered extract. Accordingly, the ratio of raw Schizandra to powdered Schizandra extract is 4:1. Similarly, the ratio of raw Rhodiola to powdered Rhodiola extract is 20:1. Finally, the ratio of raw Eleuthrococcus to powdered Eleuthrococcus extract is 15:1.

TABLE I

| | | Example | |
|---|---|---|---|
| | | Formula | Ingredient COA |
| Ingredient | | (in mg) | description |
| 1. | Schizandra extract (4:1) | 100 | NLT 2% schizandrins |
| 2. | Cordyceps mycelium extract | 25 | NLT 0.7% adenosine |
| 3. | Rhodiola extract (20:1) | 100 | NLT 3% rosavins, NLT |
| | | | 1% salidroside |
| 4. | Eleuthrococcus (15:1) | 100 | NLT 0.8% eleutherosides |
| | | | B + E |
| 5. | Goji berry extract (10%) | 200 | 1 g extract equals to 15 g |
| | | | fresh fruit |
| 6. | Grape seed extract | 60 | Huisong GSE |
| | (extract 95%) | | |
| 7. | Maltodextrin | 1470 | |
| | (sweetener) (carb) | | |
| 8. | Stevia (sweetener) | 50 | Huisong stevia |
| 9. | Fona flavoring | 150 | |
| 10. | Juice | 1800 | 1 g concentrated juice |
| | | | equals to 15 g fresh fruit |
| 11. | Evaporated Cane Sugar | 1560 | |
| | | sucrose = | |
| | | none | |
| | TOTAL | 5605 | |

There is no limitation on how the composition/mixture may be prepared. In one non-limiting example, one or more steps the technique described below are carried out (in any order including the order listed):

Step 1: Raw materials of main ingredients are converted to processed materials in a standardized extraction process.
Step 2: Processed materials (including Cordyceps mycellum extract, Rhodiola extract, Eleuthrococcus, Goji berry extract, grape seed extract) are blended into a mixture, together with maltodextrin, stevia and evaporated cane sugar. The mixture is filtered twice in water between 40-60 C in temperature for 1.5 hours each cycle.
Step 3: The mixture is then moved to a spray drying chamber. Black currant juice is fed to the chamber to blend with the mixture and Shisandra extract in controlled temperature, to graduate into fine powder format.
Step 4: The fine powder is then mixed with FONA flavor agent.
In the above example, Shisandra extract was introduced in Step 3 as stated above.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed is:

1. A composition comprising at least one species of Schizandra, at least one species of Rhodiola, an eleuthero side, at least one species of Cordyceps, at least one species of Lycium, and at least one species of Ribes.

2. The composition of claim 1, wherein the composition comprises a dry powder.

3. The composition of claim 1, wherein the Schizandra comprises Schizandra chinensis, Schizandra glaucescens, Shizandra rubriflora, Schizandra rubrifolia, or any combination thereof.

5. The composition of claim 1, wherein the Schizandra is 1.5% to 2.0% by weight of the composition.

6. The composition of claim 1, wherein the Rhodiola comprises Rhodiola rosea, Rhodiola heterodonta, Rhodiola semenovii, or any combination thereof.

7. The composition of claim 1, wherein the Rhodiola is 1.5% to 2.0% by weight of the composition.

8. The composition of claim 1, wherein the Rhodiola comprises about 3% rosavins and about 1% salidroside.

9. The composition of claim 1, wherein the eleutheroside comprises Eleutherococcus baoxinensis, Eleutherococcus brachypus, Eleutherococcus cissifolius, Eleutherococcus cuspidatus, Eleutherococcus divaricatus, Eleutherococcus eleutheristylus, Eleutherococcus giraldii, Eleutherococcus henryi, Eleutherococcus higoensis, Eleutherococcus huangshanensis, Eleutherococcus hypoleucus, Eleutherococcus japonicus, Eleutherococcus lasiogyne, Eleutherococcus leucorrhizus, Eleutherococcus nanpingensis, Eleutherococcus nikaianus, Eleutherococcus nodiflorus, Eleutherococcus pilosulus, Eleutherococcus pseudosetulosus, Eleutherococcus pubescens, Eleutherococcus rehderianus, Eleutherococcus rufinervis, Eleutherococcus scandens, Eleutherococcus senticosus, Eleutherococcus seoulensis, Eleutherococcus sessiliflorus, Eleutherococcus setchuensis, Eleutherococcus setulosus, Eleutherococcus sieboldianus, Eleutherococcus simonii, Eleutherococcus spinosus, Eleutherococcus stenophyllus, Eleutherococcus trichodon, Eleutherococcus trifoliatus, Eleutherococcus verticillatus, Eleutherococcus wardii, Eleutherococcus wilsonii, Eleutherococcus xizangensis, or any combination thereof.

10. The composition of claim 1, wherein the eleutheroside is 1.5% to 2.0% by weight of the composition.

11. The composition of claim 1, wherein the Cordyceps comprises Cordyceps bassiana, Cordyceps gunnii, Cordyceps ophioglossoides, Cordyceps sinesis, Cordyceps subsessilis, Cordyceps unilateralis, or any combination thereof.

12. The beverage of claim 1, wherein the Cordyceps is 0.3% to 0.5% by weight of the composition.

13. The composition of claim 1, wherein the Lycium comprises Lycium barbarum, Lycium chinense, or any combination thereof.

14. The composition of claim 1, wherein the Lycium is 3% to 6% by weight of the composition.

15. The composition of claim 1, wherein the Ribes comprises Ribes nigrum (blackcurrant), Ribes aciculare, Ribes alpestris, Ribes alpinum, Ribes altamirani, Ribes ambiguum, Ribes americanum, Ribes aureum, Ribes bracteosum, Ribes burejense, Ribes californicum, Ribes cereum, Ribes ciliatum, Ribes coloradense, Ribes curvatum, Ribes cynosbati, Ribes diacanthum, Ribes distans, Ribes divaricatum, Ribes echinellum, Ribes emodense, Ribes fasciculatum, Ribes fragrans, Ribes gayanum, Ribes giraldii, Ribes glaciale, Ribes glandulosum, Ribes glutinosum, Ribes grossularioides, Ribes henryi, Ribes hirtellum, Ribes hudsonianum, Ribes indecorum, Ribes inebrians, Ribes inerme, Ribes irriguum, Ribes japonicum, Ribes lacustre, Ribes lasianthum, Ribes laurifolium, Ribes laxiflorum, Ribes leptanthum, Ribes lobbii, Ribes longeracemosum, Ribes luridum, Ribes malvaceum, Ribes mandschuricum, Ribes maximowiczii, Ribes menziesii, Ribes meyeri, Ribes missouriense, Ribes montigenum, Ribes moupinense, Ribes multiflorum, Ribes nevadense, Ribes nigrum, Ribes niveum, Ribes odoratum, Ribes orientale, Ribes oxyacanthoides, Ribes petiolare, Ribes petraeum, Ribes pinetorum, Ribes procumbens, Ribes pulchellum, Ribes quercetorum, Ribes roezlii, Ribes rotundifolium, Ribes rubrum, Ribes sanguineum, Ribes setosum, Ribes silvestre, Ribes speciosum, Ribes spicatum, Ribes stenocarpum, Ribes tenue, Ribes triste, Ribes ussuriense, Ribes uva-crispa, Ribes viburnifolium, Ribes vilmorinii, Ribes viscosissimum, Ribes warszewiczii, Ribes wolfii, or any combination thereof.

16. The composition of claim 1, wherein the Ribes is 25% to 40% by weight of the composition

17. The composition of claim 1, wherein the composition further comprises a sweetener.

18. The composition of claim 1, wherein the sweetener is 0.7% to 1.0% by weight of the composition.

19. The composition of claim 17, wherein the sweetener comprises an artificial sweetener, a natural sweetener, or any combination thereof.

20. The composition of claim 1, wherein the composition further comprises 20 wt % to 30 wt % maltodextrin.

21. The composition of claim 1, wherein the composition further comprises a grape seed extract.

22. The composition of claim 1, wherein the grape seed extract is 0.7% to 1.0% by weight of the composition.

23. The composition of claim 1, further comprising a flavoring agent.

24. The composition of claim 22, wherein the flavoring agent comprises a concentrated juice.

25. A beverage comprising the composition of claim 1.

26. The beverage of claim 25 wherein the beverage is a carbonated beverage.

27. The beverage of claim 25 provided in a sealed container.

28. The beverage of claim 27 wherein the sealed contained is labeled as an energy drink.
